(19) United States
(12) Patent Application Publication
(10) Pub. No.: US 2022/0024844 A1
(43) Pub. Date: Jan. 27, 2022

(54) ZIRCONIUM-BASED AND LANTHANUM-BASED ETHYLENICALLY UNSATURATED METAL SALTS

(71) Applicant: Arkema France, Colombes (FR)

(72) Inventor: Guillaume MONNIER, Verneuil En Halatte (FR)

(21) Appl. No.: 17/299,030

(22) PCT Filed: Dec. 2, 2019
(86) PCT No.: PCT/EP2019/083357

§ 371 Date: Jun. 2, 2021

(30) Foreign Application Priority Data
Dec. 3, 2018 (FR) FR18.72212

(51) Int. Cl. C07C 51/41; C08F 216/06; C08F 222/02; C08F 216/08; C08F 220/18; C08F 2/50; C08F 2/54

(52) U.S. Cl. CPC C07C 51/412; C08F 216/06; C08F 222/02; C08F 2/54; C08F 220/18; C08F 2/50; C08F 216/08

(57) ABSTRACT

An ethylenically unsaturated and (co)polymerizable metal salt soluble in an ethylenically unsaturated reactive diluent is based on zirconium or on lanthanum and on an ethylenically unsaturated hemi-ester of a dicarboxylic acid. A process for the preparation of the salt and a crosslinkable composition comprising it are also disclosed. Uses of the metal salt include optical and dental uses, uses in composite materials, in moulding compositions, in 3D printing compositions and compositions for 3D articles and for coatings or adhesives. The finished crosslinked product which results from crosslinking the crosslinkable composition comprising the metal salt is also described.

The present invention relates to an ethylenically unsaturated and (co)polymerizable metal salt soluble in an ethylenically unsaturated reactive diluent, which is based on zirconium or on lanthanum and on an ethylenically unsaturated hemi-ester of a dicarboxylic acid, to a process for the preparation of the said salt, to a crosslinkable composition comprising it and to its varied uses in materials for optical or dental applications, in composite materials, in moulding compositions, in 3D printing compositions and compositions for 3D articles, as for coatings or adhesives, and also relates to the finished crosslinked product which results therefrom.

Ethylenically unsaturated metal salts are already known, in particular the zinc or calcium salts, such as the zinc di(meth)acrylates or calcium di(meth)acrylates used as crosslinking agents in elastomers, as described in U.S. Pat. Nos. 5,314,187 or 5,506,308. The major disadvantage of these salts for a wider use is the fact that they are not soluble in a reactive diluent medium and more particularly in compositions which can crosslink either by the peroxide route or under radiation and this fact limits their use in homogeneous crosslinkable compositions (homogeneous and transparent crosslinkable composition with all the reactive components being soluble in the said composition) in the field of coatings (including varnishes, inks), adhesives, composite materials, dental compositions, 3D printing compositions, compositions for 3D articles and moulding compositions or for compositions of transparent materials for optical applications.

WO 00/075241 and more particularly EP 1 980 565 and EP 1 189 994 describe the synthesis of soluble zinc salts from Zn oxide (ZnO) and from (meth)acrylate-functionalized hemi-ester/acid. The first stage is the synthesis of the hemi-ester/acid by reaction of a cyclic anhydride with HE(M)A. The second stage is a conventional esterification reaction between the hemi-ester/acid and the zinc oxide; the water formed during the reaction is distilled in order to shift the equilibrium with an azeotropic solvent. When this reaction is complete, a (meth)acrylate monomer is added to the medium and then the solvent is removed under reduced pressure. However, the preparation method described is not sufficient to obtain other soluble metal salts, such as zirconium oxide, zirconium or lanthanum salts. Moreover, the soluble ethylenically unsaturated zinc salts described have a limited functionality (2) and are insufficient for applications in even more high-performance materials and in particular materials having a high refractive index and a high mechanical strength with a high thermal stability.

The novel ethylenically unsaturated metal salts which are soluble and copolymerizable in an ethylenically unsaturated reactive diluent or in a crosslinkable composition containing it overcome the disadvantages and deficiencies of the known metal salts of the state of the art with the additional advantages of a broad spectrum of use in homogeneous and varied crosslinkable compositions with a broad range of targeted applications as a function of the choice of the components of the said composition as a result of the performance qualities of the said metal salt and of its solubility in the said compositions.

The first subject-matter of the invention is a (co)polymerizable ethylenically unsaturated metal salt soluble in an ethylenically unsaturated reactive diluent, which salt is based on zirconium or on lanthanum and on a specific ethylenically unsaturated hemi-ester of a dicarboxylic acid. The said salt can be or be presented in the form of a homogeneous solution in at least one ethylenically unsaturated reactive diluent.

Another subject-matter of the invention is a first process for the preparation of the said metal salt with recovery of the said salt as is in the salt form in bulk (100%). A process for the preparation in the form of a solution in at least one ethylenically unsaturated reactive diluent is another subject-matter of the invention.

Another subject-matter of the invention is a crosslinkable composition comprising at least one metal salt according to the invention.

Two final subject-matters of the invention are respectively the use of the said metal salt according to the invention in crosslinkable compositions and also the finished crosslinked object which results therefrom.

Thus, the first subject-matter of the present invention is a (co)polymerizable ethylenically unsaturated metal salt which is soluble in an ethylenically unsaturated reactive diluent, which salt is a zirconium or zirconium oxide salt or a lanthanum salt with an ethylenically unsaturated hemi-ester of a dicarboxylic acid of formula (I): (X)ₙ—R1—O₂C—R—CO₂H

and optionally, in addition to the said hemi-ester (I), in the presence (or there is presence) of a saturated monocarboxylic acid of formula (II): R2CO₂H, and the said salt is defined according to one of the following formulae (III), (IV) or (V):
[(X)ₙR1—O₂C—R—CO₂—]₄₋ₓZr(—O₂CR2)ₓ (III), with x=0 or 1 or 2 or 3, in particular x=0 or 1
[(X)ₙR1—O₂C—R—CO₂—]₂₋ₓZr(═O)(—O₂CR2)ₓ (IV), with x=0 or 1 and
[(X)ₙR1—O₂C—R—CO₂—]₃₋ₓLa(—O₂CR2)ₓ (V), with x=0 or 1 or 2, in particular x=0 or 1
and
R being the residue, without the carboxyl groups, of a dicarboxylic acid of aliphatic, cycloaliphatic, aromatic or mixed structure,
R1—(X)ₙ being the residue, without OH, of an ethylenically unsaturated monoalcohol carrying n ethylenically unsaturated functional groups X chosen from (meth)acrylate (—CO₂—C(R3)═CH₂, with R3: —H or —CH₃), vinyl (—CH₂═CH₂) or allyl (—CH₂—CH₂═CH₂), with n being an integer ranging from 1 to 5 or from 1 to 3 or n being equal to 1, and
R2 being the residue, without carboxylic group, of a saturated (without ethylenic unsaturation) monocarboxylic acid chosen from:
a monoacid other than a dicarboxylic acid hemi-ester, the said monoacid being aliphatic, cycloaliphatic, heterocyclic, aromatic or mixed, or
an acid hemi-ester of a dicarboxylic acid, of formula (VI): R′1—O₂C—R—CO₂H, with, in this case, R2═R′1—O₂C—R′— and R′1 being the residue of a saturated monoalcohol of aliphatic, cycloaliphatic, aromatic or mixed structure and R′ being the residue of a dicarboxylic acid, without carboxyl groups, of saturated structure (without ethylenic unsaturation) of aliphatic, cycloaliphatic, aromatic or mixed structure, with R′ being able to be identical to R when R is of saturated structure or with R′ being able to be different from R,
   and optionally the said metal salt being in the form of a solution in at least one ethylenically unsaturated reactive diluent copolymerizable with the said salt.

According to a specific option of the said metal salt, the said dicarboxylic acid R(—CO₂H)₂ is selected from:
for aliphatic diacids: maleic, fumaric, succinic, itaconic, butanedioic, pentanedioic, hexanedioic, heptanedioic, octanedioic, nonanedioic, decanedioic or dodecanedioic acid, C₃₆ dimer fatty acids or from their mixtures, preferably from: maleic acid, succinic acid and itaconic acid
for cycloaliphatic diacids: cyclohexane-1,4-, -1,3- or -1,2-dicarboxylic acid, tetrahydrophthalic acid, norbornanedicarboxylic acids or their mixtures
for aromatic diacids: ortho-, iso- or terephthalic acid, tetrabromophthalic acid, naphthalenedicarboxylic acids, biphenyldicarboxylic acids, in particular biphenyl-2,2′-dicarboxylic acid (diphenic acid), or their mixtures.

More particularly, in the said metal salt, the said ethylenically unsaturated monoalcohol HO—R1—(X)ₙ is selected from:
for X being allyl: allyl alcohol, monoether of allyl alcohol with an aliphatic C₂ to C₃₆ diol which can be alkoxylated or an oligoether diol or an oligoester diol, diether of allyl alcohol with an aliphatic triol which can be alkoxylated or an oligoether triol or oligoester triol, triether of allyl alcohol with an aliphatic tetrol which can be alkoxylated or with an oligoether or oligoester tetrol, mono-, di- or triallyl ester derivatives of a dicarboxylic acid/allyl alcohol hemi-ester with a polyol of respective OH functionalities of 2, 3 and 4, preferably from: allyl alcohol and monoether of allyl alcohol with an aliphatic C₂ to C₃₆ diol which can be alkoxylated
for X being vinyl: vinyl alcohol, monoether of vinyl alcohol with an aliphatic C₂ to C₃₆ diol which can be alkoxylated or an oligoether diol or an oligoester diol, diether of vinyl alcohol with an aliphatic triol which can be alkoxylated or an oligoether triol or oligoester triol, triether of vinyl alcohol with an aliphatic tetrol which can be alkoxylated or with an oligoether or oligoester tetrol, mono-, di- or triallyl ester derivatives of a dicarboxylic acid/vinyl alcohol hemi-ester with a polyol of respective OH functionalities of 2, 3 and 4, and preferably from: vinyl alcohol and monoether of vinyl alcohol with an aliphatic C₂ to C₃₆ diol which can be alkoxylated
for X being (meth)acrylate: hydroxyalkyl mono(meth)acrylate with C₂-C₈ alkyl, mono(meth)acrylate of oligoether diol or of oligoester diol, di(meth)acrylate of aliphatic triol which can be alkoxylated, di(meth)acrylate of oligoether triol or of oligoester triol, tri(meth)acrylate of aliphatic tetrol which can be alkoxylated or tri(meth)acrylate of oligoether tetrol or of oligoester tetrol, penta(meth)acrylate of aliphatic hexol which can be alkoxylated or penta(meth)acrylate of oligoether hexol or of oligoester hexol, preferably from: hydroxyalkyl mono(meth)acrylate with C₂-C₈ alkyl and mono(meth)acrylate of oligoether diol or of oligoester diol.

According to one of the preferred options of the said metal salt, the said group X is a (meth)acrylate CH₂═C(R3)—CO₂— group with R3 being —H or —CH₃ and with n ranging from 1 to 5 or from 1 to 3 or with n being equal to 1,

and with the following general formulae (III), (IV) and (V) for the said salt:
[(CH₂═C(R3)—CO₂—)ₙR1—O₂C—R—CO₂—]₄₋ₓZr(—O₂CR2)ₓ (III) with x=0 or 1 or 2 or 3, in particular x =0 or 1
[(CH₂═C(R3)—CO₂—)ₙR1—O₂C—R—CO₂—]₂₋ₓZr(═O)(—O₂CR2)ₓ (IV) with x=0 or 1 and
[(CH₂═C(R3)—CO₂—)ₙR1—O₂C—R—CO₂—]₃₋ₓLa(—O₂CR2)ₓ (V) with x=0 or 1 or 2, in particular with x=0 or 1.

According to a specific option of the said metal salt, the said dicarboxylic acid of residue R is selected from aromatic dicarboxylic acids. More particularly, the said diacid is iso- and terephthalic acid and biphenyl-2,2′-dicarboxylic acid.

More particularly still, the said group X is a (meth)acrylate group and the said monoalcohol HO—R1—(X)ₙ is selected from hydroxyethyl (meth)acrylate, hydroxypropyl (meth)acrylate, hydroxybutyl (meth)acrylate, hydroxypentyl (meth)acrylate, hydroxyhexyl (meth)acrylate, diethylene glycol mono(meth)acrylate, neopentyl glycol mono(meth)acrylate, triethylene glycol mono(meth)acrylate, dipropylene glycol mono(meth)acrylate, tripropylene glycol mono(meth)acrylate, mono(meth)acrylate of polyethylene glycol of Mn<1000, mono(meth)acrylate of polypropylene glycol of Mn<1000, polytetramethylene glycol mono(meth)acrylate, hydroxylated mono(meth)acrylate of polycaprolactone of Mn<1000, monoepoxidized (meth)acrylated compound with a secondary hydroxyl, alkoxylated mono(meth)acrylated diepoxide, optionally ethoxylated and/or propoxylated trimethylolpropane di(meth)acrylate, optionally ethoxylated and/or propoxylated glycerol di(meth)acrylate, tris(2-hydroxyethyl) isocyanurate di(meth)acrylate, pentaerythritol tri(meth)acrylate, ditrimethylolpropane tri(meth)acrylate, diglycerol tri(meth)acrylate or dipentaerythritol penta(meth)acrylate and preferably the said monoalcohol HO—R1—(X)ₙ is selected from: hydroxyethyl (meth)acrylate, hydroxypropyl (meth)acrylate or hydroxybutyl (meth)acrylate.

According to a preferred option of the said metal salt, x=0 for the salts of respective formulae (III), (IV) and (V) as defined above.

According to another specific option of the said metal salt, x=1 or 2 or 3 for the said salt of formula (III), x=1 for the said salt of formula (IV) and x=1 or 2 for the said salt of formula (V) and the said monocarboxylic acid R2CO₂H is a different monocarboxylic acid from a dicarboxylic acid hemi-ester (is not a dicarboxylic acid hemi-ester) and it is chosen from:
aliphatic monoacid with R2 being a linear C₁-C₂₁ alkyl or branched C₄-C₂₁ alkyl
cycloaliphatic monoacid with R2 being a C₆ to C₁₈ cycloalkyl
aromatic monoacid with R2 being a C₆ to C₁₈ aryl
monoacid with R2 being a radical (residue) with a heterocycle comprising a heteroatom from 0, N and S and in particular the said heterocycle being chosen from thiophene, phthalimide or furan rings and more particularly the said acid R2CO2H being chosen from: thiophenecarboxylic acid, N-phthaloylglycine and furoic acid (also known as furancarboxylic acid).

According to another specific option of the said metal salt, x=1 or 2 or 3 for the said salt of formula (III), x=1 for the said salt of formula (IV) and x=1 or 2 for the said salt of formula (V) and the said monocarboxylic acid R2CO₂H is a dicarboxylic acid hemi-ester of formula (VI): R′1—O₂C—R′—CO₂H, as defined above.

The presence (if x is other than 0) of R2CO2— and its choice in the formulae of the metal salts according to the invention following respective formulae (III), (IV) and (V) can adjust the targeted properties of the said metal salt and its affinity with the components of the targeted crosslinkable composition in order to adjust a specific performance quality in the targeted application desired. R2 is thus a major structural parameter which can be modified like the parameter x, it being possible for both to be modified according to the final applicatives and performance requirements targeted for the crosslinkable composition.

The said metal salt according to the invention is more particularly in solution in at least one ethylenically unsaturated reactive diluent, chosen in particular from (meth)acrylic, vinyl or allyl monomers and their mixtures, more particularly with a functionality of ethylenically unsaturated groups ranging from 1 to 6.

The second subject-matter of the invention is a process for the preparation of the said metal salt, which process comprises the following stages:
i) preparation of the said ethylenically unsaturated hemi-ester of formula (I) and optionally of the said saturated hemi-ester of formula (VI) by respective reactions:
   of the dicarboxylic anhydride of formula R(CO)₂O with the said ethylenically unsaturated monoalcohol HO—R1—(X)ₙ, with R, R1, X and n as defined above,
   and optionally
   of the dicarboxylic anhydride R′(CO)₂O with the said saturated monoalcohol R′1—OH, with R′ and R′1 being as defined above,
ii) neutralization by a weak base in aqueous medium of the said hemi-ester of formula (I), optionally as a mixture with the said saturated monoacid (without ethylenic unsaturation) of formula (II) which can be a monoacid other than a hemi-ester or a hemi-ester according to formula (VI), the said weak base preferably being chosen from: sodium carbonate, sodium bicarbonate or tertiary amine, in order to obtain a soluble salt of the said hemi-ester (I), optionally of the said mixture with the said monoacid (II) as defined above,
iii) addition in aqueous solution with stirring as a function of the targeted salt of respective formulae (II), (IV) or (V) of respectively zirconium tetrachloride (ZrCl₄), zirconium oxydichloride (Zr(═O)Cl₂) or lanthanum trichloride (LaCl₃), with precipitation of the said salt,
iv) filtration of the said precipitated salt with washing with water, drying and recovery of the said salt in the solid bulk form.

The invention also covers a process for the preparation of the said metal salt in solution in at least one ethylenically unsaturated reactive diluent, which process comprises, in addition to stages i) and ii) as defined for the preceding process (above), the following supplementary stages iii) to vi):
iii) addition to the aqueous solution of stage ii) of an inert organic solvent (unreactive without ethylenic unsaturation) which is a solvent for the said metal salt and is immiscible with water, in order to create a two-phase medium,
iv) addition with stirring to the two-phase medium as a function of the targeted salt of respective formulae (II), (IV) or (V) of respectively zirconium tetrachloride (ZrCl₄), zirconium oxydichloride (Zr(═O)Cl₂) or lanthanum trichloride (LaCl₃), with transfer of the said metal salt formed in solution into the organic phase of the water/organic solvent two-phase medium,
v) separation by settling with removal of the aqueous phase and addition to the recovered organic phase of at least one ethylenically unsaturated reactive diluent,
vi) removal of the said inert organic solvent by evaporation under reduced pressure and recovery of the solution of the said metal salt in the said at least one reactive diluent.

Mention may be made, as suitable inert solvents, of the following: C₁-C₃ alkyl acetates, alkanes, in particular hexane or heptane, cycloalkanes, in particular cyclohexane, methylcyclohexane, methylcyclopropane or methylcyclobutane, or aromatics, in particular toluene, benzene, xylene, ethylbenzene, cumene or mesitylene, and preferably from: ethyl acetate, heptane, cyclohexane, methylcyclohexane and toluene.

Another subject-matter of the invention is a crosslinkable composition, which comprises at least one metal salt as defined above or obtained by a process as defined above according to the invention.

According to a specific option, the said composition can be crosslinked by the peroxide route and/or by the route under radiation.

More particularly still, the said composition comprises, in addition to the said salt, at least one reactive diluent copolymerizable with the said salt selected from mono- or polyfunctional (meth)acrylic, vinyl or allyl monomers, preferably mono- or polyfunctional (meth)acrylic or vinyl monomers. In particular, the said reactive diluent copolymerizable with the said salt is at least one (meth)acrylic monomer chosen from optionally alkoxylated C₁-C₁₈ alkyl (meth)acrylates, optionally alkoxylated hydroxyalkyl (meth)acrylates with C₂-C₄ alkyl, (meth)acrylate esters of alcohols with an aromatic or cycloaliphatic structure, epoxy (meth)acrylates, urethane (meth)acrylates, optionally alkoxylated poly(meth)acrylic esters of aliphatic polyols or of oligoether polyols having from 2 to 4 ether units, with a functionality ranging from 2 to 6. The term “with an aromatic or cycloaliphatic structure” means “comprising aromatic or cycloaliphatic groups”.

According to a more specific option, the said composition additionally comprises at least one ethylenically unsaturated oligomer carrying at least one ethylenically unsaturated group. More particularly, the said oligomer carries from 1 to 6 ethylenically unsaturated groups chosen from: (meth)acrylate, vinyl and allyl, preferably (meth)acrylate or vinyl. More particularly still, the said oligomer is selected from: vinyl esters, unsaturated polyesters, polyether (meth)acrylates, polyester (meth)acrylates, (meth)acrylic (meth)acrylate copolymers, epoxy (meth)acrylates, oligourethane (meth)acrylates, polycarbonate (meth)acrylates or polycarbonate urethane (meth)acrylates.

According to a more specific option, the said composition is crosslinkable by UV radiation with the presence of a photoinitiator or by an electron beam without a photoinitiator.

The said crosslinkable composition is chosen in particular from: coating composition, adhesive composition, moulding composition, composition of composite material reinforced by glass or carbon fibres, or 3D printing composition.

A subject-matter of the invention is the use of the said metal salt in crosslinkable compositions.

More particularly, the said use relates to materials having a high refractive index for optical applications, in particular for corrective ophthalmic lenses, optical prisms or composite materials having glass or carbon fibre reinforcement, materials for dental applications, 3D printing compositions, the manufacture of 3D objects and the promotion of the adhesion of coatings or of adhesives to polar substrates.

Finally, the present invention also covers a crosslinked product, which results from the use of at least one metal salt as defined above or obtained by a process as defined above or from the crosslinking of a composition as defined above according to the invention.

More particularly, as regards the said crosslinked product, it is: a coating, an adhesive, a composite material, in particular comprising glass or carbon fibre reinforcement, a moulded part, a corrective lens or an optical prism having a high refractive index, a dental part, a 3D printing or a 3D article.

The following examples are presented in order to illustrate the invention and its performance qualities and they do not in any way limit the scope of the invention, which is defined by the scope of the claims.

### EXAMPLES

### 1) Starting Materials Used

| Starting materials | Example 1 | Example 2 | Example 3 | Example 4 |
|---|---|---|---|---|
| Ethylenically | Hydroxyethyl | Hydroxyethyl | Phenyl glycidyl | Hydroxyethyl |
| unsaturated | acrylate | methacrylate | ether acrylate | methacrylate |
| monoalcohol | (HEA, Aldrich) | (HEMA, Aldrich) | (CN131B from | (HEMA, Aldrich) |
| | | | Sartomer) | |
| Acid anhydride | Phthalic anhydride | | | |
| Weak base | Sodium bicarbonate (NaHCO₃, Aldrich) | | | |
| Metal salt | Zirconyl dichloride octahydrate (ZrOCl₂•8H₂O, Alfa Aesar) | | | Lanthanum |
| | | | | trichloride |
| | | | | heptahydrate |
| | | | | (LaCl₃•8H2O, |
| | | | | Aldrich) |
| Ethylenically | Biphenylmethanol | Cumyl phenol | Biphenylmethanol | Biphenylmethanol |
| unsaturated | acrylate (HOO8 | acrylate | acrylate (HOO8 | acrylate (HOO8 |
| diluent | from KPX) | (CD590 from | from KPX) | from KPX) |
| | | Sartomer) | | |

### 2) Preparation of the Ethylenically Unsaturated Metal Salts

### Example 1 (Invention): Zirconyl Di(HEA Phthalate) Diluted in Biphenylmethanol Acrylate (HOO8 from KPX)

Stage 1: 29.71 g of hydroxyethyl acrylate (HEA, Mw 116.12 g/mol, Sigma Aldrich), 37.89 g of phthalic anhydride (Mw 148.12, Sigma Aldrich) and 0.07 g of hydroquinone methyl ether (HQME, Aldrich) are introduced into 1 l reactor with stirring and while bubbling with air (0.3 ml/H/kg). The mixture is heated and maintained at 100° C. until the final halting criterion, determined by the measurement of the acid number (AN), expressed in mg KOH/g, is reached. The final theoretical AN value expected is 212.3 mg KOH/g.
Stage 2: A solution of 21.29 g of sodium bicarbonate (NaHCO₃, Mw 84.0 g/mol, Aldrich) dissolved in 70 g of demineralized water is added over 30 minutes to the same reactor at 60° C. and while bubbling with air. Release of CO₂ is observed. At the end of the addition of the sodium bicarbonate solution, the reaction medium is milky but becomes slightly hazy homogeneous after stirring for 1 hour. The pH of the solution is measured at 7.
Stage 3: At 60° C., with stirring and while bubbling with air, 500 g of ethyl acetate are added to the medium and then a solution of 48.19 g of zirconyl dichloride octahydrate (ZrOCl₂.8H₂O, Mw 322.25 g/mol, Alfa Aesar) dissolved in 32.0 g of demineralized water is added to the two-phase medium over 15 minutes. As the addition takes place, a white precipitate is formed in the medium, which precipitate subsequently dissolves in the organic phase. At the end of the addition, with the stirrer halted, two phases are observed: a clear colourless aqueous phase and an opaque white organic phase.
Stage 4: The aqueous phase is removed in a separating funnel and the organic phase is washed once with 50 g of a 20% sodium chloride solution.
Stage 5: After paper filtration, 55 g of biphenylmethanol acrylate (H008, KPX) are added to the organic medium and then the ethyl acetate is evaporated under reduced pressure.
After evaporation of the solvent, the product obtained is a homogeneous liquid of 121.94 g, i.e. a final yield of the zirconyl di(HEA phthalate) salt of 84%. The viscosity at 25° C. of the mixture is 5400 mPa·s and with a refractive index of 1.591.

### Example 2 (Invention): Zirconyl Di(HEMA phthalate) Diluted in Cumyl Phenol Acrylate (CD590 from Sartomer)

Stage 1: 31.62 g of hydroxyethyl methacrylate (HEMA, Mw 130.14 g/mol, Sigma Aldrich), 35.98 g of phthalic anhydride (Mw 148.12, Sigma Aldrich) and 0.07 g of hydroquinone methyl ether (HQME, Aldrich) are introduced into 1 l reactor with stirring and while bubbling with air (0.3 ml/H/kg). The mixture is heated and maintained at 100° C. until the final halting criterion, determined by the measurement of the acid number (AN), expressed in mg KOH/g, is reached. The final theoretical AN value expected is 201.6 mg KOH/g.
Stage 2: A solution of 21.75 g of sodium bicarbonate (NaHCO₃, Mw 84.0 g/mol, Aldrich) dissolved in 70 g of demineralized water is added over 30 minutes to the same reactor at 60° C. and while bubbling with air. Release of CO₂ is observed. At the end of the addition of the sodium bicarbonate solution, the reaction medium is milky but becomes slightly hazy homogeneous after stirring for 1 hour. The pH of the solution is measured at 7.
Stage 3: At 60° C., with stirring and while bubbling with air, 500 g of ethyl acetate are added to the medium and then a solution of 49.22 g of zirconyl dichloride octahydrate (ZrOCl₂.8H₂O, Mw 322.25 g/mol, Alfa Aesar) dissolved in 32.0 g of demineralized water is added to the two-phase medium over 15 minutes. As the addition takes place, a white precipitate is formed in the medium, which precipitate subsequently dissolves in the organic phase. At the end of the addition, with the stirrer halted, two phases are observed: a clear colourless aqueous phase and an opaque white organic phase.
Stage 4: The aqueous phase is removed in a separating funnel and the organic phase is washed once with 50 g of a 20% sodium chloride solution.
Stage 5: After paper filtration, 84.79 g of cumyl phenol acrylate (CD590, Sartomer) are added to the organic medium and then the ethyl acetate is evaporated under reduced pressure.
After evaporation of the solvent, the product obtained is a homogeneous liquid of 155.72 g, i.e. a final yield of the zirconyl di(HEMA phthalate) salt of approximately 88%. The viscosity at 25° C. of the mixture is 4590 mPa·s and with a refractive index of 1.561.

### Example 3 (Invention): Zirconyl Di(Phenyl Glycidyl Ether Acrylate Phthalate) Diluted in Biphenylmethanol Acrylate (H008 from KPX)

Stage 1: 44.32 g of phenyl glycidyl ether acrylate (CN131B, Mw 300 g/mol, Sartomer), 35.98 g of phthalic anhydride (Mw 148.12, Sigma Aldrich) and 0.07 g of hydroquinone methyl ether (HQME, Aldrich) are introduced into 1 l reactor with stirring and while bubbling with air (0.3 ml/H/kg). The mixture is heated and maintained at 100° C. until the final halting criterion, determined by the measurement of the acid number (AN), expressed in mg KOH/g, is reached. The final theoretical AN value expected is 123.5 mg KOH/g.
Stage 2: A solution of 15.38 g of sodium bicarbonate (NaHCO₃, Mw 84.0 g/mol, Aldrich) dissolved in 70 g of demineralized water is added over 30 minutes to the same reactor at 60° C. and while bubbling with air. Release of CO₂ is observed. At the end of the addition of the sodium bicarbonate solution, the reaction medium is milky but becomes slightly hazy homogeneous after stirring for 1 hour. The pH of the solution is measured at 7.
Stage 3: At 60° C., with stirring and while bubbling with air, a solution of 29.45 g of zirconyl dichloride octahydrate (ZrOCl₂.8H₂O, Mw 322.25 g/mol, Alfa Aesar) dissolved in 25.0 g of demineralized water is added to the medium over 15 minutes. A white precipitate very rapidly forms.
Stage 4: The white precipitate is isolated by filtration and then dried overnight in an oven at 100° C. The product recovered is subsequently dissolved in 100 g of toluene. The cloudy solution is filtered through paper in order to obtain a clear and homogeneous filtrate.
Stage 5: After paper filtration, 80.0 g of biphenylmethanol acrylate (H008 from KPX) are added to the organic medium and then the toluene is evaporated under reduced pressure.
After evaporation of the solvent, the product obtained is a slightly hazy homogeneous liquid of 132.0 g, i.e. a final yield of the zirconyl di(phenyl glycidyl ether acrylate phthalate) salt of approximately 66%. The viscosity at 25° C. of the mixture is 35 000 mPa·s and with a refractive index of 1.594.

### Example 4 (Invention): Lanthanum Tri(HEMA phthalate) Diluted in Biphenylmethanol Acrylate (H008 from KPX)

Stage 1: 15.56 g of hydroxyethyl methacrylate (HEMA, Mw 130.14 g/mol, Sigma Aldrich), 17.70 g of phthalic anhydride (Mw 148.12, Sigma Aldrich) and 0.035 g of hydroquinone methyl ether (HQME, Aldrich) are introduced into 1 I reactor with stirring and while bubbling with air (0.3 ml/H/kg). The mixture is heated and maintained at 100° C. until the final halting criterion, determined by the measurement of the acid number (AN), expressed in mg KOH/g, is reached. The final theoretical AN value expected is 201.6 mg KOH/g.
Stage 2: A solution of 10.69 g of sodium bicarbonate (NaHCO₃, Mw 84.0 g/mol, Aldrich) dissolved in 100 g of demineralized water is added over 30 minutes to the same reactor at 60° C. and while bubbling with air. Release of CO₂ is observed. At the end of the addition of the sodium bicarbonate solution, the reaction medium is milky but becomes slightly hazy homogeneous after stirring for 1 hour. The pH of the solution is measured at 7.
Stage 3: At 60° C., with stirring and while bubbling with air, 350 g of ethyl acetate are added to the medium and then a solution of 49.22 g of lanthanum trichloride heptahydrate (LaCl₃.7H₂O, Mw 371.37 g/mol, Aldrich) dissolved in 50.0 g of demineralized water is added to the two-phase medium over 15 minutes. As the addition takes place, a white precipitate is formed in the medium, which precipitate subsequently dissolves in the organic phase. At the end of the addition, with the stirrer halted, two phases are observed: a clear colourless aqueous phase and a slightly cloudy organic phase.
Stage 4: The aqueous phase is removed in a separating funnel and the organic phase is washed once with 50 g of a 10% sodium chloride solution.
Stage 5: After paper filtration, 30 g of biphenylmethanol acrylate (H008 from KPX) are added to the organic medium and then the ethyl acetate is evaporated under reduced pressure.
After evaporation of the solvent, the product obtained is a homogeneous liquid of 65.8 g, i.e. a final yield of the lanthanum tri(HEMA phthalate) salt of approximately 91%. The viscosity at 25° C. of the mixture is 9200 mPa·s and with a refractive index of 1.588.

### 3) Characteristics of the Prepared Salts

| | Soluble metal | | | | |
|---|---|---|---|---|---|
| | salt/dilution | | | | Refractive |
| | monomer | Appearance | Brookfield | | index |
| Exam- | (reactive | of the | viscosity | | of the |
| ples | diluent) ratio | mixture | @25° C. | | mixture |
| Exam- | 55/45 | Clear | 5400 | mPa · s | 1.591 |
| ple 1 | | homogeneous | | | |
| Exam- | 45/55 | Clear | 4590 | mPa · s | 1.561 |
| ple 2 | | homogeneous | | | |
| Exam- | 60/40 | Slightly hazy | 35 000 | mPa · s | 1.594 |
| ple 3 | | homogeneous | | | |
| Exam- | 55/45 | Clear | 9200 | mPa · s | 1.588 |
| ple 4 | | homogeneous | | | |

1. A (Co)polymerizable ethylenically unsaturated metal salt which is soluble in an ethylenically unsaturated reactive diluent, wherein the metal salt is a zirconium or zirconium oxide salt or a lanthanum salt with an ethylenically unsaturated hemi-ester of a dicarboxylic acid of formula (I):
(X)ₙ—R1—O₂C—R—CO₂ (I)
and optionally, in addition to the said hemi-ester (I), in the presence of a saturated monocarboxylic acid of formula (II): R2CO₂H, and that the said salt is defined according to one of the following formulae (Ill), (IV) or (V):
[(X)ₙR1—O₂C—R—CO₂—]₄₋ₓZr(—O₂CR2)ₓ (III), with x=0 or 1 or 2 or 3,
[(X)ₙR1—O₂C—R—CO₂—]₂₋ₓZr(=O)(—O₂CR2)ₓ (IV), with x=0 or 1, and
[(X)ₙR1—O₂C—R—CO₂—]₃₋ₓLa(—O₂CR2)ₓ (V), with x=0 or 1 or 2,
R being the residue, without the carboxyl groups, of a dicarboxylic acid of aliphatic, cycloaliphatic, aromatic or mixed structure,
R1—(X)ₙ being the residue, without OH, of an ethylenically unsaturated monoalcohol carrying n ethylenically unsaturated functional groups X chosen from (meth)acrylate (—CO₂—C(R3)═CH₂, with R3: —H or —CH₃), vinyl (—CH₂═CH₂) or allyl (—CH₂—CH₂═CH₂), with n being an integer ranging from 1 to 5 or from 1 to 3 or n being equal to 1, and
R2 being the residue, without carboxylic group, of a saturated (without ethylenic unsaturation) monocarboxylic acid chosen from:
a monoacid other than a dicarboxylic acid hemi-ester, the said monoacid being aliphatic, cycloaliphatic, heterocyclic, aromatic or mixed, or
an acid hemi-ester of a dicarboxylic acid, of formula (VI): R′1—O₂C—R′—CO₂H,
with, in this case, R2═R′1—O₂C—R′—
and
R′1 being the residue of a saturated monoalcohol of aliphatic, cycloaliphatic, aromatic or mixed structure, and
R′ being the residue of a dicarboxylic acid, without carboxyl groups, of saturated structure (without ethylenic unsaturation) of aliphatic, cycloaliphatic, aromatic or mixed structure, which can be identical to R when R is of saturated structure or which can be different from R,
and optionally the said metal salt being in the form of a solution in an ethylenically unsaturated reactive diluent copolymerizable with the said salt.

2. The metal salt according to claim 1, wherein the dicarboxylic acid R(—CO₂H)₂ is selected from:
for aliphatic diacids: maleic, fumaric, succinic, itaconic, butanedioic, pentanedioic, hexanedioic, heptanedioic, octanedioic, nonanedioic, decanedioic or dodecanedioic acid, C₃₆ dimer fatty acids or from their mixtures
for cycloaliphatic diacids: cyclohexane-1,4-, -1,3- or -1,2-dicarboxylic acid, tetrahydrophthalic acid, norbornanedicarboxylic acids or their mixtures
for aromatic diacids: ortho-, iso- or terephthalic acid, tetrabromophthalic acid, naphthalenedicarboxylic acids, biphenyldicarboxylic acids, in particular biphenyl-2,2′-dicarboxylic acid (diphenic acid), or their mixtures.

3. The metal salt according to claim 1, wherein the ethylenically unsaturated monoalcohol HO—R1—(X)ₙ is selected from:
for X being allyl: allyl alcohol, monoether of allyl alcohol with an aliphatic C₂ to C₃₆ diol which can be alkoxylated or an oligoether diol or an oligoester diol, diether of allyl alcohol with an aliphatic triol which can be alkoxylated or an oligoether triol or oligoester triol, triether of allyl alcohol with an aliphatic tetrol which can be alkoxylated or with an oligoether or oligoester tetrol, mono-, di- or triallyl ester derivatives of a dicarboxylic acid/allyl alcohol hemi-ester with a polyol of respective OH functionalities of 2, 3 and 4
for X being vinyl: vinyl alcohol, monoether of vinyl alcohol with an aliphatic C₂ to C₃₆ diol which can be alkoxylated or an oligoether diol or an oligoester diol, diether of vinyl alcohol with an aliphatic triol which can be alkoxylated or an oligoether triol or oligoester triol, triether of vinyl alcohol with an aliphatic tetrol which can be alkoxylated or with an oligoether or oligoester tetrol, mono-, di- or triallyl ester derivatives of a dicarboxylic acid/vinyl alcohol hemi-ester with a polyol of respective OH functionalities of 2, 3 and 4
for X being (meth)acrylate: hydroxyalkyl mono(meth)acrylate with C₂-C₈ alkyl, mono(meth)acrylate of oligoether diol or of oligoester diol, di(meth)acrylate of aliphatic triol which can be alkoxylated, di(meth)acrylate of oligoether triol or of oligoester triol, tri(meth)acrylate of aliphatic tetrol which can be alkoxylated or tri(meth)acrylate of oligoether tetrol or of oligoester tetrol, penta(meth)acrylate of aliphatic hexol which can be alkoxylated or penta(meth)acrylate of oligoether hexol or of oligoester hexol.

4. The metal salt according to claim 1, wherein characterized in that the group X is the (meth)acrylate CH₂═C(R3)—CO₂— group with R3 being —H or —CH₃ and with n ranging from 1 to 5 or from 1 to 3 or with n being equal to 1,
and with the following general formulae (III), (IV) and (V) for the said salt:
[(CH₂═C(R3)—CO₂—)ₙR1—O₂C—R—CO₂—]₄₋ₓZr(—O₂CR2)ₓ (III) with x=0 or 1 or 2 or 3,
[(CH₂═C(R3)—CO₂—)ₙR1—O₂C—R—CO₂—]₂₋ₓZr(═O)(—O₂CR2)ₓ (IV) with x=0 or 1 and
[(CH₂═C(R3)—CO₂—)ₙR1—O₂C—R—CO₂—]₃₋ₓLa(—O₂CR2)ₓ (V) with x=0 or 1 or 2,

5. The metal salt according to claim 1, wherein the dicarboxylic acid of residue R comprises aromatic dicarboxylic acids.

6. The metal salt according to claim 5, wherein the dicarboxylic acid of residue R is iso- and terephthalic acid and biphenyl-2,2′-dicarboxylic acid.

7. The metal salt according to claim 1, wherein the group X is the (meth)acrylate group and the monoalcohol HO—R1—(X)ₙ is selected from the group consisting of hydroxyethyl (meth)acrylate, hydroxypropyl (meth)acrylate, hydroxybutyl (meth)acrylate, hydroxypentyl (meth)acrylate, hydroxyhexyl (meth)acrylate, diethylene glycol mono(meth)acrylate, neopentyl glycol mono(meth)acrylate, triethylene glycol mono(meth)acrylate, dipropylene glycol mono(meth)acrylate, tripropylene glycol mono(meth)acrylate, mono(meth)acrylate of polyethylene glycol of Mn<1000, mono(meth)acrylate of polypropylene glycol of Mn<1000, polytetramethylene glycol mono(meth)acrylate, hydroxylated mono(meth)acrylate of polycaprolactone of Mn<1000, monoepoxidized (meth)acrylated compound with a secondary hydroxyl, alkoxylated mono(meth)acrylated diepoxide, optionally ethoxylated and/or propoxylated trimethylolpropane di(meth)acrylate, optionally ethoxylated and propoxylated glycerol di(meth)acrylate, tris(2-hydroxyethyl) isocyanurate di(meth)acrylate, pentaerythritol tri(meth)acrylate, ditrimethylolpropane tri(meth)acrylate, diglycerol tri(meth)acrylate and dipentaerythritol penta(meth)acrylate.

8. The metal salt according to claim 1, wherein x=0 for the salts of respective formulae (III), (IV) and (V).

9. The metal salt according to claim 1, wherein x=1 or 2 or 3 for the salt of formula (III), that x=1 for the salt of formula (IV) and x=1 or 2 for the salt of formula (V) and the monocarboxylic acid R2CO₂H is a different monocarboxylic acid from a dicarboxylic acid hemi-ester and is selected from the group consisting of:
aliphatic monoacid with R2 being a linear C₁-C₂₁ alkyl or branched C₄-C₂₁ alkyl
cycloaliphatic monoacid with R2 being a C₆ to C₁₈ cycloalkyl
aromatic monoacid with R2 being a C₆ to C₁₈ aryl and
monoacid with R2 comprising a heterocycle comprising a heteroatom from O, N and S and in particular from thiophene, phthalimide or furan rings and more particularly from: thiophenecarboxylic acid, N-phthaloylglycine and furoic acid (also known as furancarboxylic acid).

10. The metal salt according claim 1, wherein x=1 or 2 or 3 for the salt of formula (III), x=1 for the salt of formula (IV) and x=1 or 2 for the salt of formula (V) and the monocarboxylic acid R2CO₂H is a dicarboxylic acid hemi-ester of formula (VI): R′1—O₂C—R′—CO₂H.

11. The metal salt according to claim 1, wherein the metal salt is in solution comprising the ethylenically unsaturated reactive diluent, chosen from (meth)acrylic, vinyl or allyl monomers and their mixtures.

12. A process for the preparation of the metal salt as defined according to claim 1, comprising the following stages:
i) preparing the ethylenically unsaturated hemi-ester of formula (I) and optionally of the said saturated hemi-ester of formula (VI) by respective reactions:
of the dicarboxylic anhydride of formula R(CO)₂O with the said ethylenically unsaturated monoalcohol HO—R1—(X)ₙ,
and optionally of the dicarboxylic anhydride R′(CO)₂O with the said saturated monoalcohol R′1—OH,
ii) neutralizing by a weak base in aqueous medium the hemi-ester of formula (I), optionally as a mixture with the saturated monoacid (without ethylenic unsaturation) of formula (II) which can be a monoacid other than a hemi-ester or a hemi-ester according to formula (VI),
in order to obtain a soluble salt of the hemi-ester (I), optionally of the mixture with the monoacid (II) as defined above,
iii) adding in aqueous solution with stirring as a function of the targeted salt of respective formulae (II), (IV) or (V) of respectively zirconium tetrachloride (ZrCl₄), zirconium oxydichloride (Zr(═O)Cl₂) or lanthanum trichloride (LaCl₃), with precipitation of the salt,
iv) filtering of the precipitated salt with washing with water, drying and recovery of the salt in the solid bulk form.

13. A process for the preparation of the metal salt according to claim 1, wherein the process is done in solution comprising the ethylenically unsaturated reactive diluent, wherein the process comprises the following stages comprises
i) preparing the ethylenically unsaturated hemi-ester of formula (I) and optionally of the said saturated hemi-ester of formula (VI) by respective reactions:
of the dicarboxylic anhydride of formula R(CO)₂O with the ethylenically unsaturated monoalcohol HO—R1—(X)ₙ,
and optionally of the dicarboxylic anhydride R′(CO)₂O with the said saturated monoalcohol R′1—OH,
ii) neutralizing by a weak base in aqueous medium the hemi-ester of formula (I), optionally as a mixture with the saturated monoacid (without ethylenic unsaturation) of formula (II) which can be a monoacid other than a hemi-ester or a hemi-ester according to formula (VI),
iii) adding to the aqueous solution of stage ii) an inert organic solvent (unreactive without ethylenic unsaturation) which is a solvent for the metal salt and is immiscible with water, in order to create a two-phase medium,
iv) adding with stirring to the two-phase medium as a function of the targeted salt of respective formulae (II), (IV) or (V) respectively zirconium tetrachloride (ZrCl₄), zirconium oxydichloride (Zr(═O)Cl₂) or lanthanum trichloride (LaCl₃), with transfer of the metal salt formed in solution into the organic phase of the water/organic solvent two-phase medium,
v) separation by settling with removal of the aqueous phase and addition to the recovered organic phase of at least one ethylenically unsaturated reactive diluent,
vi) removal of the inert organic solvent by evaporation under reduced pressure and recovery of the solution of the metal salt in the at least one reactive diluent.

14. A crosslinkable composition comprising at least one metal salt as defined according to claim 1.

15. A composition according to claim 14, wherein the composition is adapted to be crosslinked by the peroxide route and/or by the route under radiation.

16. A composition according to claim 14, further comprising, in addition to the at least one metal salt, at least one reactive diluent copolymerizable with the metal salt selected from mono- or polyfunctional (meth)acrylic, vinyl or allyl monomers.

17. A composition according to claim 16, wherein the reactive diluent is at least one (meth)acrylic monomer selected from the group consisting of optionally alkoxylated C₁-C₁₈ alkyl (meth)acrylates, optionally alkoxylated hydroxyalkyl (meth)acrylates with C₂-C₄ alkyl, epoxy (meth)acrylates, urethane (meth)acrylates, optionally alkoxylated poly(meth)acrylic esters of aliphatic polyols or of oligoether polyols having from 2 to 4 ether units, with a functionality ranging from 2 to 6.

18. A composition according to claim 14, further comprising at least one ethylenically unsaturated oligomer carrying at least one ethylenically unsaturated group.

19. A composition according to claim 18, wherein the oligomer carries from 1 to 6 ethylenically unsaturated groups chosen from: (meth)acrylate, vinyl and allyl, preferably (meth)acrylate or vinyl.

20. A composition according to claim 18, wherein the oligomer is selected from the group consisting of: vinyl esters, unsaturated polyesters, polyether (meth)acrylates, polyester (meth)acrylates, (meth)acrylic (meth)acrylate copolymers, epoxy (meth)acrylates, oligourethane (meth)acrylates, polycarbonate (meth)acrylates and polycarbonate urethane (meth)acrylates.

21. A composition according to claim 14, wherein the composition is crosslinkable by UV radiation with the presence of a photoinitiator or by an electron beam without a photoinitiator.

22. A composition according to claim 14, wherein the composition is chosen from: a coating composition, an adhesive composition, a moulding composition, a composition of composite material reinforced by glass or carbon fibres, or a 3D printing composition.

23. A method of using the composition according to claim 14 comprising crosslinking the composition.

24. (canceled)

25. A crosslinked product resulting from crosslinking the composition according to claim 14.

26. A product according to claim 25, wherein the product is: a coating, an adhesive, a composite material, a moulded part, a corrective lens or an optical prism having a high refractive index, a dental part, a 3D printing or a 3D article.
